SEQUENCE LISTING (3 sequences)

SEQ ID NO: 1 - DNA, 998 bp, Triticum aestivum
  CDS 25..660
accagcaccaagaaaacaaaaagcatgaagatgatgagcaccagggccctcgcgctcggcgcggctgccgtcctcgccttcgccgcggcgaccgctcaggcccagaggtgcggcgagcaaggcagcaacatggagtgccccaacaacctctgctgcagccagtacggctactgcgggatgggcggcgactactgcggcaagggctgccagaacggcgcctgctggaccagcaagcgctgcggcagccaggccggcggcgcgacgtgcaccaacaaccagtgctgcagccagtacgggtactgcggcttcggcgccgagtactgcggcgccggctgccagggcggcccctgccgcgccgacatcaagtgcggcagccaggccggcggcaagctgtgcccgaacaacctctgctgcagccagtggggattctgcggcctcggttccgagttctgcggcggcggctgccagagcggtgcttgcagcaccgacaaaccgtgcggcaaggacgccggcggcagagtttgcactaacaactactgttgtagcaagtggggatcctgtggcatcggcccgggctattgcggtgcaggctgccagagtggcggctgcgatggtgtcttcgccgaggccatcaccgccaactccactcttctccaagaatgatgatcaatcttgctatggcagtattgcaacgacgaataatccgtggcaatctcattgccacctacggtttcccttgacttacttttagagtactagtccttaataattctctagcttgcaatatgatgtgcaggttactgcagcagaaacaaaatattgctgtcgtgcatgcatggaaatattgcagtgagaaagtactgtgtggcaatatagggtgtgctattgttgccgcaaattagttttcttgttatgacctgttgtcaggatgcatgcatggctgttgtaatgttggagtacttcgtgatttcgttgcaatatattaccatggttctcac

SEQ ID NO: 2 - DNA, 3935 bp, Mus musculus
  exon 1369..1423
  intron 1424..1576
  exon 1577..1701
  intron 1702..2650
  exon 2651..2767
gcttaactggtttcctgaaaggtatcttggagataggaacagactctcagagcatggtcagaaagccacagctcatcaatgaaatggtcagggacttcctgtcctgctccatgcataaatgaaagacgaagacaactcaaattggcatttgaggggcagataaacaggagcatccggtagtttcacaggtggtcgggtagcaggagccgggttggttggttggtctgtggagagtgcagggattaagggaagaggcctggaccccaacttcttccttggctacccccctgaaaatgtcacctgccttgcatggacgaactcacaggcaggaatgggttggcttgggtggggacatcctgcaggttccaccctcatgttggttcatcttcaacattgtactgacttcttcccacttgacattcctcaaggtcctgtgatcatggctgggtctagtgaggttcaaacctgcactgccctacccacacccacacccagctcagcgtcagtcaggatcaacaattacctagagatcatctttctggggcttaagcattggtgggagcagatgggatatgagctggggatttgggaatgggggaagatatctgctccccctccccctacaccctagccttttaaaaggccttctcaggtcagagaccaggagaaaagtataggagagatacacaatggaccaggaagaagaaaagggagagggaggctcagaccttctagacaaggtaagagggctctggctgactccaccatccgcttcttgaggtctcggcacctgtaattgacaagattaattcatttatagggcatctaattagcaagcaagtctctggagtcccctgacccagttactataacacacagggggtataggtaggagagtataagagcccctcctcagggcaaatgaatggattcttagtactgtcccccaagagatagtaggtactaggatttaggggcacttctgagccccatttccctggtaagtgtcccaaccccccaaatcaacccaagcctggtctcaatctaggacagtggtagaatgctgtccctagagtcagtaccatgtgaaattgtgctgcaggcaggggccccaggctgggaggtgggggttgggggagtcagggcaggtcagggaaggagactcaggtttcatttagagaaattctgcagacccgtgaggactatggtgagagcagagatgggaaggcaggcactgtttcgggtggatgctgtctggaagacagggaaggcacagaccaaactaaaccaatcacgtctgtccccaaggcaggttcaccggaccaggaaggcttcttcaacctgctgacccacgtgcagggcgatcggatggaggagcagcgctgttccttgcaggctgggccaggccagaacccagaaagccgtaagcagggcgtgattgggccgtgtgtgtgtgtgtgtgtgtgtgtgtgtgtgtgtgtgtgtgtggcaggagtgctggggttctgggatcttgtggatcttgggactcaggatggggtctgtattcatgcctgcctgtctctgctccaagcagagggtggccctgctccagagatggacaatctcatggatatgctggtcaacacccagggccgccgcatggacgaccagcgtgtaacagttaattccctgcctggcttccaacctatcggccccaaggtaggtgatgtccagattacctgtgagactccacatagctctctaaatctatgacctgtctctaggcaggaaaggagaggaccctatgaacacgtaaagtgctatgggcttaaggtcaggtggcaggactcatgctagtgcagaactatggctggaaattacagttcctgctccaacatctgtatatttgggagaggccacagggagaaaacaggcagttttcctggaaggcatatgaatgcatacccctataaatcaatgaagagtagggcttctgtttgggagtgttttgctttattgtttttgagacagggtttcatgtagctctggctggcatgttctcctacatgtgcatcctgggttctgggataacaggtgtgagtcaccatgagtgatgtatgtgggtagggatagaacccagggctttgatgcagtctctatcaactgagctccagccccagccctatgtctgtgtacattagcatacatgtttagagctccgggcacacgtgtgcacacgcaggtggaggccagaagtcaatctcctgccctgggagctttcagtgccctggaactccaggtagatcaggctctctagctaggaagcccttgggatcctcctgactcttaagcactgagattacaagtgcataaacccacacctggcttaaactcaggtcttcaaatgagcatagcaaggatttcaatgactgagctatcttctcaactcaactgtttgtttgtttgttttagtatttagctttgaactcaaaataatcctcctgcctgtttcttgagtactgggattacaggtatacactaacaggccaatgtctgaccaaataccaccaccctaattagcagacgaaaaaaaaacattgtttggaggcacttctgacttgcactttccttggtcccctccctccgtctgacccttcttcatccccaggatggaatgcagaaacgacctgggaccctcagccctcaacccctgctcacccctcaggatcctgctgcactcagcttccgcaggaacagcagcccccagccccagacacaagctccttgagagttctagccatcctgggcctcccactggcccctgaaaacaataaaacacttggcactagcaacaaagagttgagtgtgtgttattttctgtggtggggaagggagctgggacttgaggaactgaaggtctcaggagctctgctgggcagcttgaagaagtctctcttctttctgcttccggatcttctgcttaaattcttctagctcctggcgctggaatggggaaaggggtgtgatgggaaggaaggaagagtacaggcctcacagcctggactcactcacactatcctccctttggcttcagagttcagtatccacactgggagccccatgccaatcacaatcactgtacaagtgagttcagcttcatccctcggggaaaaggtaatatgtgacaccatttgtgccctcccctctttttaagatggggtctcatatactacaggctagccttgagctcaccaggcggcagagaatagccagaattctcaatcctcttgcatccatctcctgagtgctggaatgctggaattacagcttcctctcctgtctccctctctctatccccatgcagcccaggctagcttcaatctgatactcctcctactcctccttccaagtgtccgtaggtatacaccatcacaaacaacaagaaacctttatggagacaaggtctctagcccaggctagtctggaattcctactcagtctgctgcttccactttcctacctatggctgagggtgaaatctttattccaagcccaactaggtaagagtgactcagctccttggggaaaacaggttactgacctgaccctccttctctcttggccacagctccctctgtggaacaaagtcacaggtgagaacacaaggcaggagaatccagagccccacatccacaacagggttgactcatgagaggcagacaatggatctcaatagcaagttggtgcttcataccctcccttccgcaggaattatccatcaagcactttgatacccaccttacgctggacaacatagtcctcaaaccactcagcctgattggagatccagaacataaccacggggaaagtgaggtagagggacatctgtaaaagcagaggtgggtggagcacagggagattgcagggaagcccaaaggacaggtccggagctc

SEQ ID NO: 3 - DNA, 3279 bp, Mus musculus
  CDS 891..1379
atctctgtctccaccactcagaggcactcacagactccagttctgccatctgtccacatacactgcctgggttccacctcccactgacattcccttgtaggtccccagcttcttccctggcctcacgtctcccatgggaggtggaggatcagtttaggcggaatggctggtaggattttggtggacgtgagagccaatcctgtggctatgtggttggatcgatcaaaccacggcctctgggagccgagccagccgtctgtctggcagatgatttgggatttgagagctgcaggttcagatgggaggtgacagtgggctgggtcctgatggtgataaaggagagggagacaccagggcacctgacaggacctgacaggggctatgacagagtggggtggggggtgcggaggaggaggcaaccatggaaagttggcttggctgactacagaaaactgaaatgtgtgccaccggtgctaccccgccctgccacctctttcctggacagtcttcggttacctccatgtgtctataacctcacctatctcccaacagcgctgtggagtattccattcttcacaaacaagcaaagctccagcttgccactaccactgtagtcaaggtggttgccacagcagttgatatcagtgctctggtccccagggagcccatcaccctccagcctgcctacagcacagctttaccagttaggaggcagttggacacacacactcctgtgtcccctgttctgagaactgggtggggccagaaaggctggaaagggaggcgggccttcaggtggcctcttctcttggcatcggaggatccagcccacttgattccctgacgctggtggtagtggtggcagtggcaatcgctgtagcacttgggccatggcagaggatgggccgcagaagcagcagctggagatgccgctggttctggaccaggacctgacccagcagatgcggctccgagtagagagcctgaagcagcgtggggagaagaagcaggatggtgagaagctgatccggccggctgagtccgtctaccgcctcgatttcatccagcagcagaagctgcagttcgatcactggaacgtggttctggacaagcccggcaaggtcaccatcacgggcacctcgcagaactggacgcccgacctcaccaacctcatgacacgccagctgctggaccccgccgccatcttctggcgcaaggaagactccgacgccatggattggaatgaggcagacgccctggagtttggggagcgcctttctgatctggccaagatccgcaaggtcatgtatttcctcatcacctttggcgagggcgtggagcctgccaacctaaaggcctctgtggtgtttaaccagctctgatgacagccctggctgccctacccctggccccacctctcccttgcctggatctccttcctcatgtgtatttgggggacattcttctagctgctcctcctgtgctcatcttggccagagttcccccgagtgctacatcccctccttttccctggtgccagtgctgcggctcacagtgatgtcccatggctccgtagtctagatctagaagccggatgctgctactatagactgtagaggccttttgggtccacgtgggaagatggatgggccccctgtggtgaagagcgggactgagagataaagagactgaccaagagatgcaaacggccagcactgattcctcccttcagggacgggagactgagactggacaggaacaccttccggggaacctggcaagaaggcgtttgccctgctggccaaagctggagccaggaggcgaatgcccagcctctggcagcaggaaggttctcctcccagtgtcggcagcagcccgctgtgaccttagggccttcaagacactgggcaggatgacagcggggcttgatctgactgcttttccaggtctgggcctggtttttatggagaagtgagagagtgtgtagaaactgaaacaactctagccacccacgctcatatgggtattgagagatggcataactatttgtatggatgtgggcctgagggctagtcttggtgaggagtaaggctaactttagtttaattattgagctggtactggcttgtgggcttggtggaggtgatcctgactgaggcgtccttggtgcagtgctttttgaactgggagactgagactcgaatggtgtagcagagttagaggggtccagggctctgagctagcaacagtgatgtccctgttaggaaggctggcatttgctgctcgctggtgttgtgccctgctgtcacccccctgggcatatcctggctgttctcctggagtgcagacccctaagtaaggcttgggtgggggcagttaggatgcctgacgtctgaagtgggctggagctatctgactgtgatgcctaaactgacaggaaaacggtggcacagttagcaggttcagctctaccccaagtctcattgtccctcgccttgcacatcctgaaagccttccattgcctgttacctagcatcagccagaggtacctcagcagtgtcccctgactgtctcaaggctgcctccctcgggcatactgaaggtaggatctgtcccagctggtgagctgccaggactgcaaaccccagctcaggtgcaggattctggaggcaggagataggctgtggtaccggtgtctcttgagccggtgcctctgctccataacatgcttgccgaagcactggccggtgcttctggattctgctgactctagggagccacacccagacagtgcctctgcctttctgcttctcttcctgacctctccctacagctttagagacccctttggttcacactgcctgtgccccaactctgcctcactcggatccgtctgccctgtggggacatgagtgtctctgttgtgcctgtttcacaataaagactgtgtgccctcccctctgtggtgtggtgtgtgtgcctccgtggtgtggtttgcacatcttgctgcaagcccatagcatcagaatccttctctcatgggccctgtagctctgagcaactccaccctgccagccttgaggatgaggccgagtcgtgagatctctcatgaggattgagtttcacctgtcagccaggtttcctggctgccctgcaggtaccaatcctctagggtatgaaagagcatgctaaagctatgcttggggcaggggagtgtagcgggtaggactgatactaatttagcttggtcttggtcactgtttggctgtgccctctaga

(19) United States
(12) Patent Application Publication
(10) Pub. No.: US 2004/0073960 A1
(43) Pub. Date: Apr. 15, 2004

(54) Model animals for visualization of neutral pathways

(75) Inventor: Yoshihiro Yoshihara, Osaka (JP)

Correspondence Address: DAVIDSON, DAVIDSON & KAPPEL, LLC, 485 SEVENTH AVENUE, 14TH FLOOR, NEW YORK, NY, 10018, US

(73) Assignee: Chugai Seiyaku Kabushiki Kaisha, Tokyo (JP)

(21) Appl. No.: 10/620,148

(22) Filed: Jul. 14, 2003

Related U.S. Application Data
(63) Continuation of application No. 09/763,117, filed on Feb. 15, 2001, now abandoned.
Application No. 09/763,117 is a 371 of international application No. PCT/JP99/04439, filed on Aug. 18, 1999.

(30) Foreign Application Priority Data
Aug. 19, 1998 (JP) 232817/1998

(51) Int. Cl. A01K 67/00; A61K 31/00

(52) U.S. Cl. 800/8; 514/1

(57) ABSTRACT

The present invention provides a transgenic animal into which a gene encoding a trans-synaptic tracer protein is introduced so as to direct specific expression in particular neurons. The use of this transgenic animal permits the selective visualization of functional neural pathways through a particular group of neurons, which could not have been achieved by tracing technique using a conventional trans-synaptic tracer protein.

### FILED OF THE INVENTION

The present invention relates to transgenic animals into which a gene encoding a trans-synaptic tracer protein is introduced so as to direct specific expression in particular types of neurons, a method for screening neuromimetic substances using the transgenic animals, and a neuromimetic substance obtainable by the screening method.

### PRIOR ART

The brain with its various functions including learning, memory, multisensory recognition and integration, motor development and control, as well as emotion is composed of complex, but well-ordered neural networks. To study brain structure and function, there is a need to understand the molecular mechanisms for formation, maintenance and plasticity of neural pathways. In particular, it is undoubtedly the touchstone of studies in these various areas of neuroscience to elucidate how neurons extend their axons in the correct direction, how they recognize target cells, how they form synapses, how they form and maintain neural networks, and how they further plastically change the formed neural pathways as needed.

A variety of plant lectins have been conventionally used as trans-synaptic tracers in neuroanatomical studies on neuronal connectivity. In particular, wheat germ agglutinin (WGA) has been most efficiently transferred from primary neurons to secondary neurons across synapses, thereby exhibiting its usefulness in any neural systems. In the visual system, for example, WGA injected into one eye is taken up by ganglion cells of the retina and then transported through optic nerves to the lateral geniculate nucleus of the thalamus, where WGA is trans-synaptically transferred to thalamic secondary neurons, resulting in a WGA-labeled visual cortical area which is the projection site of the thalamic secondary neurons. In this way, the ocular dominance columns can be visualized. Thus, the technique using WGA as a tracer is very useful and powerful, and has greatly contributed to the development of neuroscience.

However, the above conventional tracing technique using WGA does not allow selective visualization of functional neural pathways through a particular group of neurons because WGA was taken up by all the cells surrounding the site of WGA injection. In addition, other problems have also been pointed out, for example, serious immune responses induced in a WGA-injected animal due to the recognition of WGA as a foreign substance.

### PROBLEMS TO BE SOLVED BY THE INVENTION

The tracing technique using WGA is very useful for studying functional connectivity patterns between neurons, but it also involves the various problems mentioned above. The object of the present invention is to overcome these problems.

### MEANS FOR SOLVING THE PROBLEMS

Our research efforts were directed to overcoming the above problems, and we have found that these problems can be overcome by using a transgenic animal into which a gene encoding a trans-synaptic tracer protein is introduced so as to direct specific expression in particular neurons, thereby finally completing the invention.

Thus, the present invention provides transgenic animals into which a gene encoding a trans-synaptic tracer protein is introduced so as to direct specific expression in particular neurons.

The present invention also provides a method for screening neuromimetic substances, which comprises administering a test substance to the transgenic animal mentioned above, and selecting a neuromimetic substance from among the test substances by using as an indicator the trans-synaptic tracer protein expressed in the animal's neurons.

The present invention further provides a neuromimetic substance obtainable by the screening method mentioned above.

This specification includes part or all of the contents as disclosed in the specification and/or drawings of Japanese Patent Application No. 10-232817 which is a priority document of the present application.

### DISCLOSURE OF THE INVENTION

The present invention will now be described in more detail.

The transgenic animal of the present invention is characterized in that a gene encoding a trans-synaptic tracer protein is introduced so as to direct specific expression in particular neurons.

In addition to WGA mentioned above, examples of the trans-synaptic tracer protein include, but are not limited to, Concanavalin A agglutinin (ConA), Pisum Sativum agglutinin (PSA), Lens Culinaris agglutinin (LCA) and the like. To express a gene encoding a trans-synaptic tracer protein in particular neurons, a promoter specific to the particular neurons may be connected upstream of the gene, but any other technique can be used for this purpose. The promoter specific to particular neurons includes, but is not limited to, cerebellar Purkinje cell-specific L7 promoter, olfactory receptor cell-specific OMP promoter and the like. As used in the transgenic animals of the present invention, the term “specific” or “specifically” means that the trans-synaptic tracer protein is sufficiently expressed to distinguish particular neurons from other cells when it is visualized with an enzyme-labeled antibody etc., but it does not necessarily mean that no trans-synaptic tracer protein gene is expressed in any other cell.

A wild-type gene encoding the trans-synaptic tracer protein may be used without any further modification. For the WGA gene, the modified gene that lacks a C-terminal propeptide-coding segment may be preferably used for the reason described below.

Any animal having neural pathways may be used in the present invention.

The transgenic animal of the present invention may be constructed as follows. A fragment containing the promoter specific to particular neurons and a fragment containing the trans-synaptic tracer protein gene may be amplified by PCR, respectively, and these amplified fragments may then be inserted into an existing vector for recombination. The resulting recombinant vector may be injected into fertilized eggs or embryos from recipient animals. A transgenic animal having the trans-synaptic tracer protein gene may be selected out of the resulting animals. For the L7 promoter, the fragment containing the promoter specific to particular neurons may be obtained by preparing primers that can amplify a region upstream from the initiation codon of the nucleotide sequence shown in SEQ ID NO: 2; amplifying a part of the promoter region by PCR using mouse genomic DNA as a template; and then screening a mouse genomic DNA library (e.g., commercially available mouse Genomic DNA Library SC945301 (Stratagene)) using the amplified PCR product as a probe. For the OMP promoter, such a fragment may be obtained by preparing a primer that can amplify a region upstream from the initiation codon of the nucleotide sequence shown in SEQ ID NO: 3; and carrying out PCR using mouse genomic DNA as a template. For the WGA gene, the fragment containing the trans-synaptic tracer protein gene may be obtained by preparing a primer that can amplify a coding-region of the nucleotide sequence shown in SEQ ID NO: 1; and carrying out PCR using wheat germ cDNA as a template.

The transgenic animal of the present invention is useful for the elucidation of causes for various neurogenic diseases and the establishment of medical treatment for these diseases. For example, the transgenic animal of the present invention may be crossed with an animal model for diseases resulting from abnormal neural pathways or with a spontaneously mutated animal model. The resulting animal may then be used to analyze the abnormal neural pathways responsible for the diseases or compensatory pathways induced by the diseases. The transgenic animal of the present invention may also be used to create an artificial pathological model for Parkinson's disease, ischaemia, head injury or various mental diseases for the analysis of injured pathways or compensatory pathways. Further, the transgenic animal of the present invention developing pathological conditions may be administered with various drugs in order to assess the potency of the administered drugs, i.e., their ability to restore injured pathways or to form compensatory pathways, by using trans-synaptic tracer protein as an indicator.

In addition, the transgenic animal's tissues expressing the trans-synaptic tracer protein may be primarily cultured to create cultured neurons expressing the trans-synaptic tracer protein, which may then be used for the screening of drugs that affect cell survival and maintenance, dendrite extension, synapse formation, various enzymatic activities, and/or neurotransmitter production.

The neuromimetic substance of the present invention may be obtained according to the above screening methods, for example, by screening test substances such as peptides, proteins, non-peptide compounds, synthetic compounds, fermented products from microorganisms, marine organism extracts, plant extracts, cell extracts, or animal tissue extracts. These test substances may be novel or known compounds.

The present invention will be further described in the following example. The example is provided for illustrative purposes only, and is not intended to limit the scope of the invention.

### EXAMPLES

### Example 1

### Construction of a Vector Expressing WGA

A wild-type WGA cDNA insert (1.0 kb) excised from pWGA-D (Smith, J. J. & Raikhel, N. V., Plant Mol. Biol. 13, 601-603 (1989)) was blunt-ended, followed by addition of a BstX I site. This insert was subcloned into a BstX I site of a mammalian expression vector pEF-BOS (Mizushima, S. & Nagata, S., Nucl. Acids Res. 18, 5322 (1990)) to construct a plasmid pEF-WGA. Mouse nueroblastoma N2a cells were transfected with this plasmid using Lipofectamine and Opti-MEM (Gibco/BRL). After 48 hours of transfection, the cells were tested for the presence of expressed WGA by Western blotting using anti-WGA antibody. As shown in FIG. 2, WGA was detected (lane 2, MW 24 kD), but it had a significantly larger size than that of an authentic WGA (lane 4, MW 18 kD).

Since the plant WGA has a C-terminal propeptide (15 amino acid residues) which is involved in selective delivery of the lectin into vacuoles (Broadwell, R. D. & Balin, B. J., J. Comp. Neurol. 242, 632-650 (1985)), the difference in molecular weight was thought to be caused by the absence of C-terminal propeptide-processing mechanism in animal cells. Thus, we decided to construct a plasmid containing a DNA segment encoding a truncated WGA which lacks the C-terminal propeptide.

A plasmid pEF-tWGA containing the truncated WGA-coding cDNA (FIG. 1(2)) was constructed from the plasmid pEF-WGA by replacing a codon GTC (valine 198) in the wild-type WGA-coding DNA (FIG. 1(1)) with an opal stop codon TGA by PCR mutagenesis using not-completely complementary primers. The N2a cells were transfected with this plasmid pEF-tWGA and then tested for the presence of expressed WGA by Western blotting, thereby detecting the truncated WGA having the same size as the authentic WGA (FIG. 2, lane 3). In addition, the amount of the truncated WGA produced was significantly larger than that of the wild-type WGA. Thus, we decided to use this truncated WGA-coding DNA in all the following experiments.

The N2a cells transfected with pEF-tWGA were treated with anti-WGA polyclonal antibody and Cy3 anti-rabbit antibody IgG (Jackson) (10 μg/ml, Sigma), followed by observation using a confocal laser scanning microscopy system (Bio-Rad MRC-600) equipped with Zeiss Axiophot F1 microscope (FIG. 3). As shown in FIG. 3, the truncated WGA strongly bound to the intracellular granule-like structures of N2a cells.

### Example 2

### Construction of a pL7-tWGA-Introduced Mouse

A mouse L7 promoter region (3.5 kb) was amplified from Pcp2-z06 plasmid (Vandaele, S. et al., Genus Dev. 5, 1136-1148 (1991)). L7 (Pcp2) gene promoter has been analyzed in detail (Oberdick, J. et al., Neuron 1, 367-376 (1988); Oberdick, J. et al., Science 248, 223-226 (1990); Oberdick, J. et al., Neuron 10, 1007-1018 (1993); Vandaele, S. et al., Genus Dev. 5, 1136-1148 (1991)), and used for cerebellar Purkinje cell-specific expression of foreign genes (Feddersen, R. M. et al., Neuron 9, 955-966 (1992); Burright, E. N. et al., Cell 82, 937-948 (1995)). This amplified fragment was subcloned into a blunt-ended BamH I site of pBstN vector, which contains human β-globin gene introns and SV 40 polyadenylation signal. A tWGA cDNA sequence (0.6 kb) excised from pEF-tWGA was ligated to a blunt-ended EcoR I site of the pBstN vector to construct a plasmid pL7-tWGA for cerebellar Purkinje cell-specific expression of the truncated WGA (FIG. 1(3)).

The purified pL7-tWGA was injected into the male pronucleus of fertilized eggs from FVB/N mice (CLEA Japan), mainly according to the procedures described by Nohmi, T. et al., Environment. Mol. Mutagenesis 28, 465-470 (1996). The pL7-tWGA-injected eggs were cultured and transferred into the oviduct of ICR pseudopregnant recipients (CLEA Japan). Tail samples taken for DNA analysis were screened for the integrated transgene by PCR and Southern analysis, thereby obtaining a transgenic mouse having the full-length transgene.

The presence of expressed WGA mRNA and protein in this transgenic animal's brain was determined by in situ hybridization and immunohistochemistry, respectively.

In situ hybridization was carried out according to the procedures described by Yoshihara, Y. et al., J. Neurosci. 17, 5830-5842 (1997) as follows.

Sections (50 μm) of an adult mouse brain perfused with paraformaldehyde were treated with proteinase K (10 μg/ml at 25° C. for 30 min), acetylated, dehydrated, and then air-dried. An antisense riboprobe for WGA (540 nucleotides in length) was prepared using ³⁵S-UTP (Amersham) and an RNA transcription kit (Stratagene). The sections were hybridized overnight with the above antisense riboprobe (1×10⁶ cpm/ml) in a humidified chamber at 56° C. After hybridization, the sections were washed with 4×SSC, treated with RNase A (10 μg/mil at 37° C. for 30 min), washed with 0.05×SSC, dehydrated with ethanol, and then exposed to βmax X-ray film (Amersham).

Also, immunohistochemistry was carried out as follows.

Sections (50 μm) of a mouse brain perfused with paraformaldehyde were cut with a sliding microtome, pre-treated with 0.3% H₂O₂, blocked, and then incubated for 2 to 24 hours at room temperature with anti-WGA polyclonal antibody (3 μg/ml, Sigma) which had been absorbed with 1% acetone powder of mouse brain. The sections were then incubated either with biotin anti-rabbit IgG (Zymed), followed by a Vectastain ABC elite kit (Vector), or with horseradish peroxidase anti-rabbit IgG (Jackson). The generated signals were visualized through the Ni²⁺-enhanced diaminobenzidine/peroxide reaction for analysis using a transmission microscopy system.

FIG. 4(1) shows WGA mRNA detection in a section including the whole brain tissue. FIG. 4(2) shows WGA protein detection in an adjacent section to the section shown in FIG. 4 (1). As shown in both figures, WGA mRNA detection is limited to the Purkinje cells, while the WGA protein is expressed not only in the Purkinje cells, but also in other cells anatomically and functionally associated with the Purkinje cells. FIG. 5 shows WGA protein detection in a section including the cerebellum, indicating that the WGA protein is expressed in the deep cerebellar nuclei (dentate, fastigial, interposed) and the vestibular nucleus, as well as the Purkinje cells.

Axons of the Purkinje cells form synapses with neurons in the deep cerebellar nuclei (Ito, M., The cerebellum and neural control., New York, Raven Press (1984); Altman, J. & Bayer, S. A., Development of the cerebellar system: in relation to its evolution, structure, and functions., Boca Raton, Fla., CRC Press (1996)). Since some Purkinje cells directly project to the vestibular nucleus, secondary neurons are also present in the vestibular nucleus. In view of the foregoing, WGA is thought to be transported to secondary and tertiary neurons of the Purkinje cells.

Double immunofluorescence labeling was used to determine the presence of Purkinje cells and expressed WGA protein in the cerebellum of the transgenic mouse transformed with pL7-tWGA. The Purkinje cells were detected by anti-calbindin antibody (Sigma) and FITC anti-mouse IgG (Cappel). Calbindin is specifically found in the Purkinje cells. The WGA protein was detected by anti-WGA antibody and Cy3 anti-rabbit IgG (Jackson). FIGS. 6(1) and (2) show WGA protein and Calbindin in the deep cerebellar nuclei, respectively. FIG. 6(3) shows the detection of both. These figures indicate the trans-synaptic transfer of WGA protein from axon termini of the Purkinje cells to neurons in the deep cerebellar nuclei.

Immunohistochemistry with anti-WGA antibody was used to determine the presence of expressed WGA protein in brain parts other than the cerebellum of the transgenic mouse transformed with the plasmid pL7-tWGA. FIGS. 7(1) and (2) show WGA protein detection in a section including the thalamic ventrolateral nucleus and in a section including the red nucleus, respectively. FIGS. 7(3) and (4) show magnified views of FIGS. 7(1) and (2), respectively. FIGS. 7(5) to (8) show WGA protein detection in a section including the superior colliculus, in a section including the gigant cellular reticular nucleus, in a section including the vestibular nucleus, and in a section including the inferior olivary nucleus, respectively. These figures indicate that the WGA protein is detected in any of the thalamic ventrolateral nucleus, red nucleus, superior colliculus, gigant cellular reticular nucleus, vestibular nucleus and inferior olivary nucleus. All of these neurons form synapses with axons from the deep cerebellar nuclei, and correspond to tertiary neurons of the Purkinje cells.

### Example 3

### Construction of a pOMP-tWGA-Introduced Mouse

An OMP promoter region (0.9 kb; Buiakova, O. I. et al., Genomics 20, 452-462 (1994)) was amplified by PCR from mouse genomic DNA and subcloned into a blunt-ended BamH I site of pBstN vector (FIG. 1(4)). A tWGA cDNA sequence was inserted downstream of the OMP promoter region to obtain a plasmid pOMP-tWGA. This plasmid pOMP-tWGA was used to construct a transgenic mouse, as described in Example 2.

The presence of expressed WGA protein in the vomeronasal organ of the above transgenic mouse was determined by an immunofluorescence labeling technique using anti-WGA antibody and Cy3 anti-rabbit IgG. FIGS. 9(1) and (2) show WGA protein detection in a section including the vomeronasal organ and a magnified view thereof, respectively, indicating that the WGA protein is highly expressed in the vomeronasal epithelium and nerve bundles thereof.

Double immunofluorescence labeling was used to determine the presence of axons and expressed WGA protein in the vomeronasal organ of the transgenic mouse transformed with pOMP-tWGA. The axons were detected by anti-NCAM antibody and FITC anti-mouse IgG (Cappel). NCAM is specifically found in the axons. The WGA protein was detected by anti-WGA antibody and Cy3 anti-rabbit IgG (Jackson). FIGS. 10(1) and (2) show WGA protein detection and axon detection, respectively. FIG. 10(3) shows the detection of both. These figures indicate that NCAM is evenly expressed in the olfactory and vomeronasal nerves, while the WGA protein is highly expressed in the vomeronasal nerves, in particular.

Immunohistochemistry was used to determine the presence of expressed WGA protein in the brain of the transgenic mouse transformed with pOMP-tWGA.

FIG. 11 shows WGA protein detection in a section including the whole brain, indicating that the WGA protein is highly expressed in the accessory olfactory bulb.

FIGS. 12(1) and (2) show WGA protein detection in a section including the accessory olfactory bulb, indicating that the WGA protein is expressed not only in vomeronasal axon termini of the glomerulus, but also in external plexiform layer and granule cell layer thereof.

FIGS. 13(1), (2) and (3) show WGA protein detection in a section including the lateral olfactory tract, indicating that the WGA protein is also expressed in mitral/tufted cell axons of the lateral olfactory tract, which corresponds to axons of secondary neurons.

FIGS. 14(1) and (4) show WGA protein detection in a section including the medial amygdaloid nucleus. FIGS. 14(2) and (3) show WGA protein detection in a section including the posteromedial cortical amygdaloid nucleus and in a section including the bed nucleus of stria terminalis, respectively. These figures indicate that the WGA protein is expressed in any of the medial amygdaloid nucleus, posteromedial cortical amygdaloid nucleus, and bed nucleus of stria terminalis, which correspond to tertiary neurons of the vomeronasal cells.

All publications, patents and patent applications cited herein are incorporated herein by reference in their entirely.

### EFFECTS OF THE INVENTION

The present invention permits the selective visualization of functional neural pathways through a particular group of neurons, which could not have been achieved by tracing technique using a conventional trans-synaptic tracer protein. Further, the present invention does not have any of the problems observed in the tracing technique using a conventional trans-synaptic tracer protein, for example, serious immune responses caused by injection of the trans-synaptic tracer protein into an animal and individual differences due to injection technique.

### BRIEF DESCRIPTION OF THE DRAWINGS

FIG. 1 shows the construction of WGA gene.

FIG. 2 shows Western blotting of the WGA gene product (electrophoresis photograph).

FIG. 3 shows WGA protein detection in N2a cells (microphotograph).

FIG. 4 shows WGA mRNA detection and WGA protein detection in a section of the brain (microphotographs).

FIG. 5 shows WGA protein detection in a section including the cerebellum (microphotograph).

FIG. 6 shows WGA protein detection in the deep cerebellar nuclei (microphotographs).

FIG. 7 shows WGA protein detection in various brain sections (microphotographs).

FIG. 8 shows a schematic diagram of neural pathways originating from Purkinje cells.

FIG. 9 shows WGA protein detection in a section including the vomeronasal organ (microphotographs).

FIG. 10 shows axon detection and WGA protein detection in a section including axons of the vomeronasal organ (microphotographs).

FIG. 11 shows WGA protein detection in a section of the brain (microphotograph).

FIG. 12 shows WGA protein detection in a section including the accessory olfactory bulb (microphotographs).

FIG. 13 shows WGA protein detection in a section including the lateral olfactory tract (microphotographs).

FIG. 14 shows WGA protein detection in a section including the medial amygdaloid nucleus, posteromedial cortical amygdaloid nucleus, and bed nucleus of stria terminalis (microphotographs).

FIG. 15 shows a schematic diagram of neural pathways originating from vomeronasal sensory neurons.

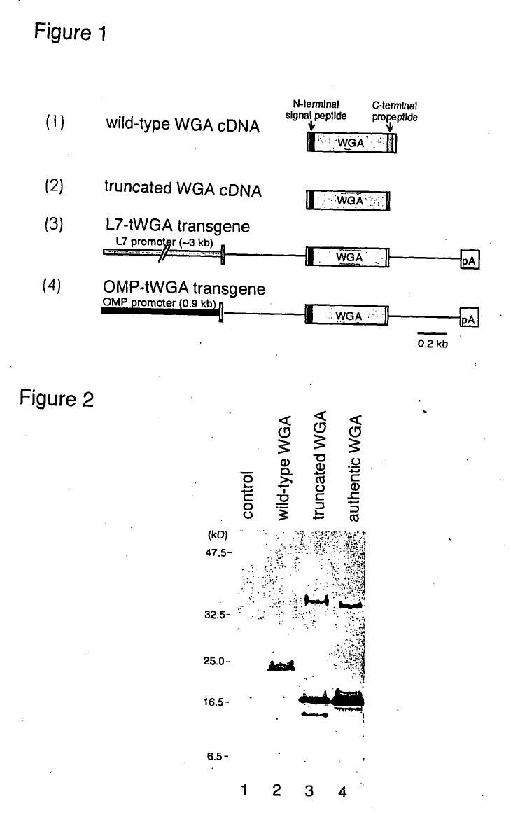

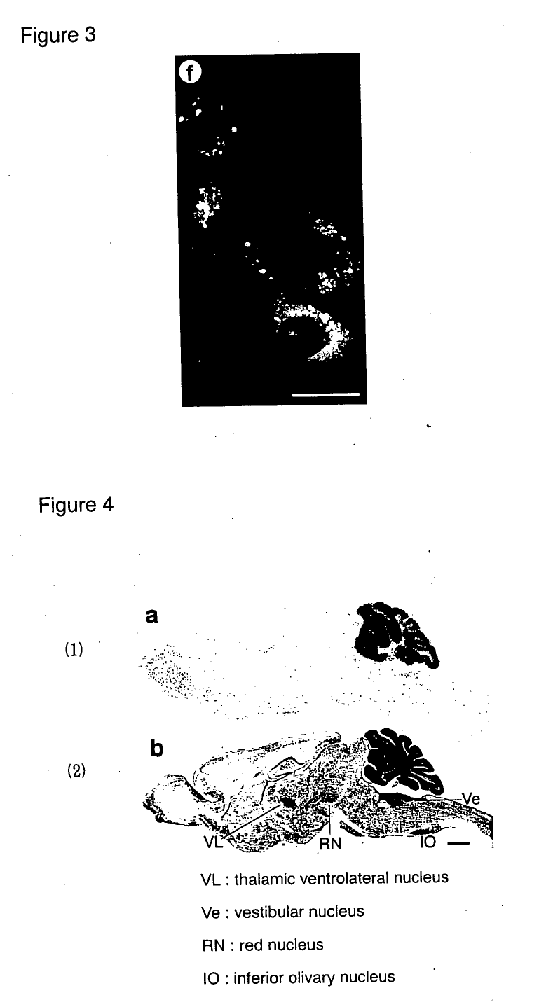

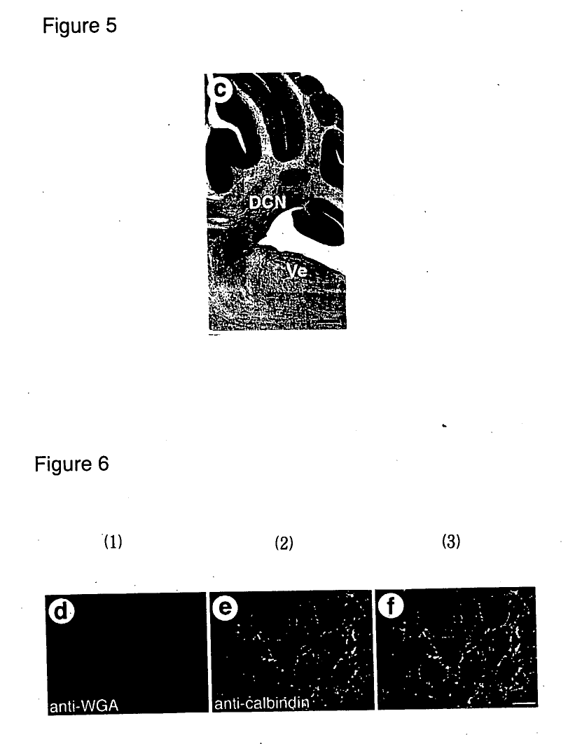

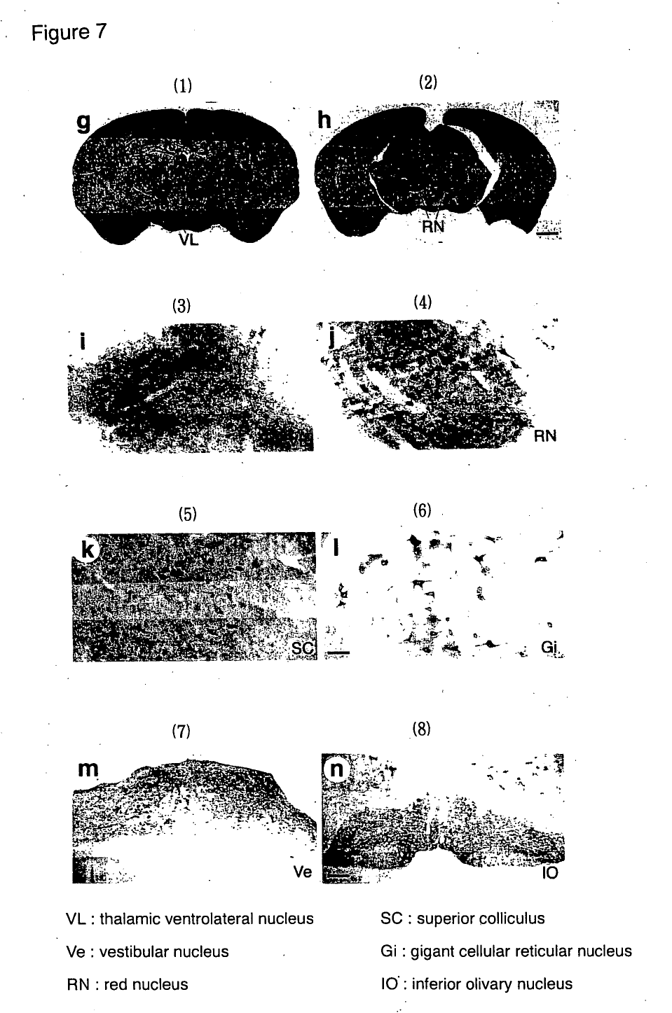

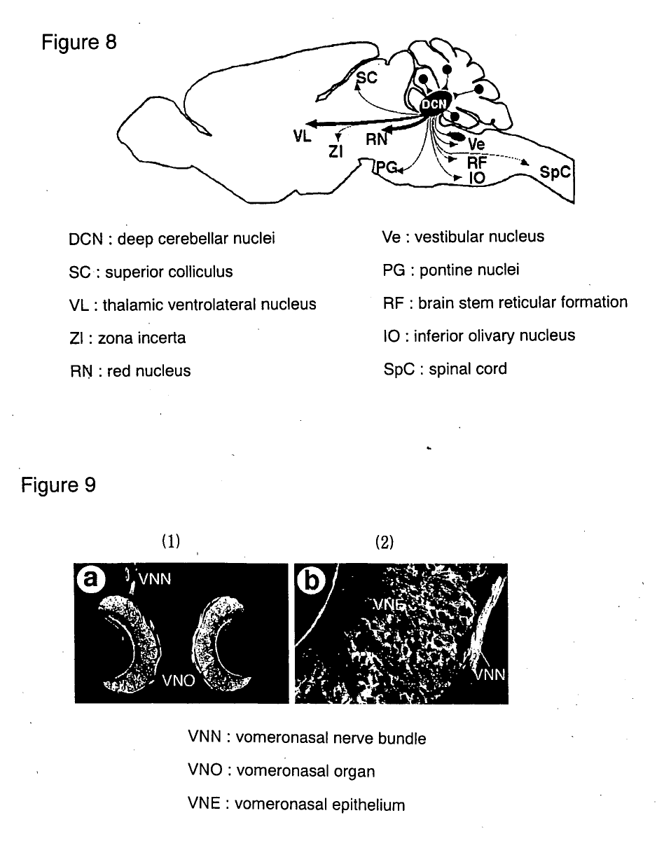

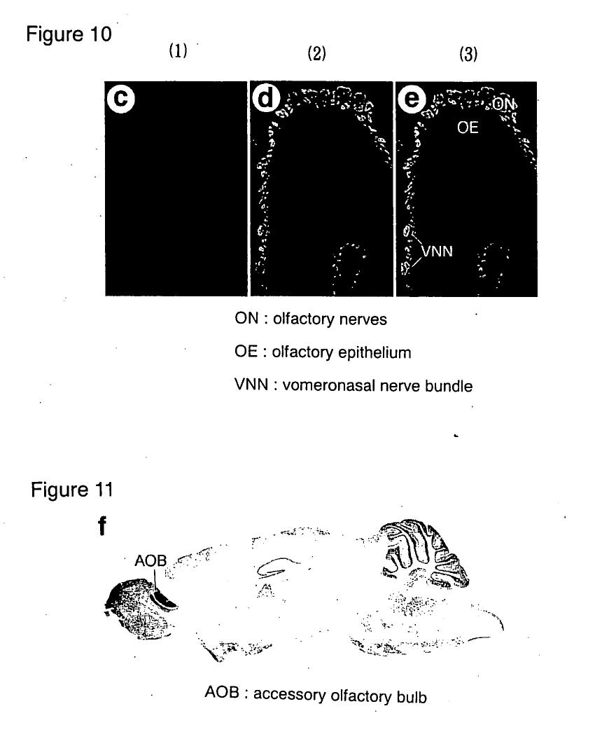

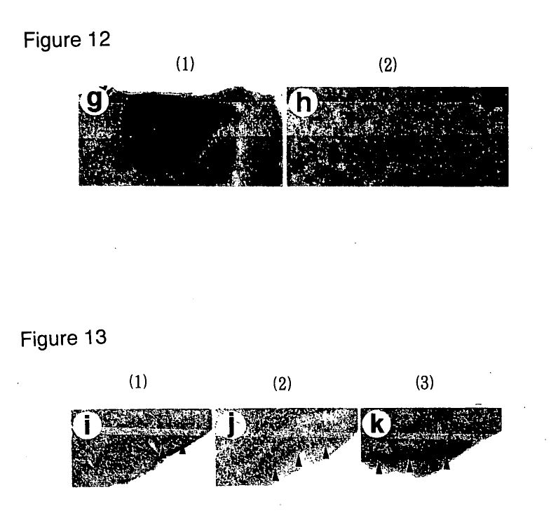

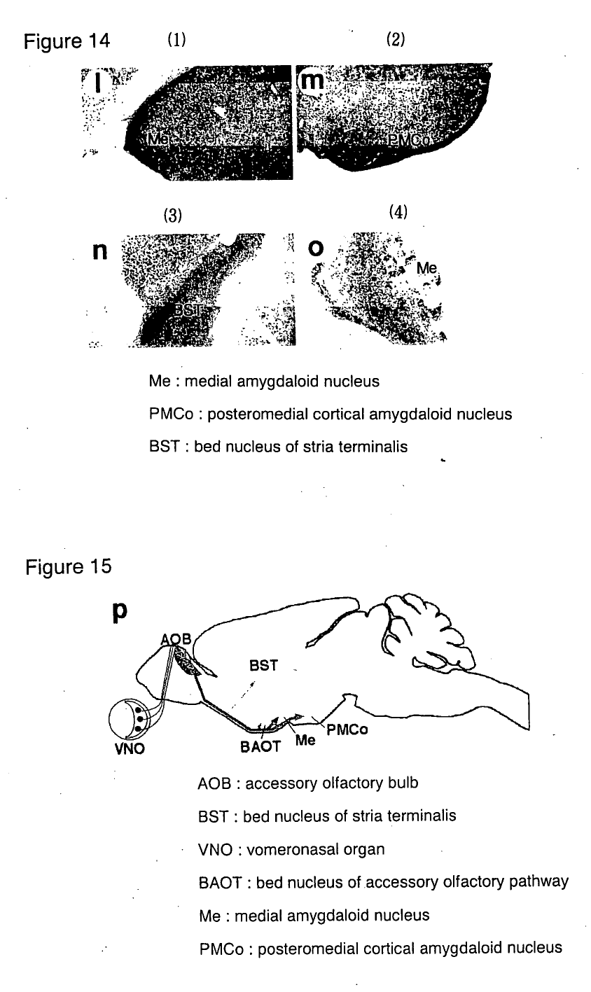

1. A transgenic animal into which a gene encoding a trans-synaptic tracer protein is introduced so as to direct specific expression in particular neurons.

2. The transgenic animal according to claim 1, wherein a promoter specific to the particular neurons is located upstream of the gene encoding the trans-synaptic tracer protein.

3. The transgenic animal according to claim 1 or 2, wherein the trans-synaptic tracer protein is wheat germ agglutinin.

4. A method for screening neuromimetic substances, which comprises:
administering a test substance to the transgenic animal according to any one of claims 1 to 3; and
selecting a neuromimetic substance from among the test substances by using as an indicator the trans-synaptic tracer protein expressed in neurons of the transgenic animal.

5. A neuromimetic substance obtainable by the screening method according to claim 4.
